(19) United States
(12) Patent Application Publication
(10) Pub. No.: US 2025/0345031 A1
(43) Pub. Date: Nov. 13, 2025

(54) ULTRASONIC ENDOSCOPE

(71) Applicant: FUJIFILM Corporation, Tokyo (JP)

(72) Inventors: Karin HATTORI, Kanagawa (JP); Yasuhiko MORIMOTO, Kanagawa (JP); Masaya INOUE, Kanagawa (JP)

(73) Assignee: FUJIFILM Corporation, Tokyo (JP)

(21) Appl. No.: 19/189,294

(22) Filed: Apr. 25, 2025

(30) Foreign Application Priority Data
May 10, 2024 (JP) 2024-077271

(51) Int. Cl. A61B 8/12; A61B 1/00; A61B 1/05; A61B 8/00

(52) U.S. Cl. CPC A61B 8/12; A61B 1/00082; A61B 1/00098; A61B 1/05; A61B 8/445

(57) ABSTRACT

An ultrasonic endoscope includes: a distal end portion disposed on a distal end side of an insertion part extending along a longitudinal axis direction; an ultrasonic transducer configured to emit an ultrasonic wave toward one side in a first direction orthogonal to the longitudinal axis direction; and an imaging unit disposed on a base end side with respect to the ultrasonic transducer. The distal end portion includes a first region having the ultrasonic transducer; and a second region disposed on the base end side of the first region and having the imaging unit. Bottom surface portions of the first and second regions are each disposed on the other side in the first direction opposite to the one side, and the bottom surface portion of the first region is disposed on the other side in the first direction with respect to the bottom surface portion of the second region.

### CROSS-REFERENCE TO RELATED APPLICATION

The present application claims priority under 35 U.S.C § 119(a) to Japanese Patent Application No. 2024-077271 filed on May 10, 2024, which is hereby expressly incorporated by reference, in its entirety, into the present application.

### BACKGROUND OF THE INVENTION

### 1. Field of the Invention

The present invention relates to an ultrasonic endoscope, and more particularly to an ultrasonic endoscope comprising an ultrasonic transducer provided at a distal end portion of an insertion part.

### 2. Description of the Related Art

As the ultrasonic endoscope, an ultrasonic endoscope is known which comprises a convex type ultrasonic transducer at a distal end portion of an insertion part, in which a treatment tool outlet port is disposed on a base end side of the ultrasonic transducer at the distal end portion (for example, see WO2020/179909A).

In addition, an imaging unit for observing a treatment target part and an illumination unit for emitting illumination light toward the treatment target part are provided at the distal end portion of the insertion part, in addition to the ultrasonic transducer.

### SUMMARY OF THE INVENTION

In the convex type ultrasonic endoscope, the imaging unit is configured as an oblique-viewing endoscope disposed at a position at which the optical axis is oblique to an insertion direction of the distal end portion. That is, since the insertion direction and a viewing direction (optical axis direction) do not match each other, it is easy to press the ultrasonic endoscope toward a subject. Therefore, the viewing direction close to a direct vision is desired.

On the other hand, in a case in which the optical axis of the imaging unit is inclined in a direct vision direction, a proportion (area) of the ultrasonic transducer reflected in an observation viewing range increases (so-called “vignetting” problem), and it is difficult to insert the ultrasonic endoscope. In addition, in order to avoid the vignetting caused by the ultrasonic transducer, it is also considered to simply change the disposition of the ultrasonic transducer so that a proportion (area) of the reflected light in the observation viewing range is small, but a diameter of the ultrasonic endoscope becomes large.

The present invention has been made in view of such circumstances, and an object of the present invention is to provide an ultrasonic endoscope that can bring an optical axis of an imaging unit closer to a direct vision direction and contributes to reduction in diameter.

A first aspect relates to an ultrasonic endoscope comprising: a distal end portion that is disposed on a distal end side of an insertion part extending along a longitudinal axis direction; an ultrasonic transducer that is provided at the distal end portion and that emits an ultrasonic wave toward one side in a first direction orthogonal to the longitudinal axis direction; and an imaging unit that is disposed on a base end side in the longitudinal axis direction with respect to the ultrasonic transducer and that images a subject, in which the distal end portion includes a first region in which the ultrasonic transducer is provided and a second region that is disposed on the base end side in the longitudinal axis direction with respect to the first region and in which the imaging unit is provided, a first bottom surface portion of the first region and a second bottom surface portion of the second region are each disposed on the other side in the first direction, which is opposite to the one side, and the first bottom surface portion is disposed on the other side in the first direction with respect to the second bottom surface portion.

A second aspect relates to the ultrasonic endoscope according to the first aspect, in which, in a case of being viewed in a second direction orthogonal to each of the longitudinal axis direction and the first direction, an angle formed between a first central axis of the first region and a second central axis of the second region is 1 degree or less.

A third aspect relates to the ultrasonic endoscope according to the second aspect, in which, in a case of being viewed in the second direction, an angle formed between a plane direction of the first bottom surface portion and the longitudinal axis direction is 1 degree or less.

A fourth aspect relates to the ultrasonic endoscope according to any one of the first to third aspects, in which the distal end portion includes a balloon groove that is provided between the first region and the second region and that is used for attaching a balloon that covers the ultrasonic transducer.

A fifth aspect relates to the ultrasonic endoscope according to the fourth aspect, in which a groove region of the balloon groove on the other side in the first direction is formed of a pair of groove side surface portions facing each other in the longitudinal axis direction, a first groove side surface portion that is one of the pair of groove side surface portions is provided on a base end side of the first region in the longitudinal axis direction, and a second groove side surface portion that is the other of the pair of groove side surface portions is provided on a distal end side of the second region in the longitudinal axis direction, and the first groove side surface portion is formed to extend toward the other side in the first direction with respect to the second groove side surface portion.

A sixth aspect relates to the ultrasonic endoscope according to any one of the first to fifth aspects, in which the second region includes an elevator unit in which an elevator is provided in a rotationally movable manner.

A seventh aspect relates to the ultrasonic endoscope according to the sixth aspect, in which the elevator unit includes the elevator and a case member that defines a space portion in which the elevator is housed.

An eighth aspect relates to the ultrasonic endoscope according to the seventh aspect, in which the case member includes a treatment tool outlet port that is open to the space portion, and the case member is provided with a pipe member that communicates with the treatment tool outlet port.

A ninth aspect relates to the ultrasonic endoscope according to the eighth aspect, further comprising: an imaging unit housing portion that houses the imaging unit, in which, in a case of being viewed in the first direction, at least a part of a region of the imaging unit housing portion is disposed at a position overlapping with the elevator unit.

A tenth aspect relates to the ultrasonic endoscope according to the ninth aspect, in which, in a case of being viewed in a second direction orthogonal to each of the longitudinal axis direction and the first direction, at least a part of a region of the imaging unit housing portion is disposed at a position overlapping with the pipe member.

An eleventh aspect relates to the ultrasonic endoscope according to the ninth or tenth aspect, in which, in a case of being viewed in a second direction orthogonal to each of the longitudinal axis direction and the first direction, a position of an outermost end portion side of the imaging unit housing portion on the other side in the first direction is provided on the other side in the first direction with respect to a position of an outermost end portion side of the pipe member on the one side in the first direction.

According to the present invention, it is possible to bring the optical axis of the imaging unit closer to the direct vision direction and to achieve the reduction in diameter.

### BRIEF DESCRIPTION OF THE DRAWINGS

FIG. 1 is an overall configuration diagram of an ultrasonic endoscope according to the present embodiment.

FIG. 2 is a perspective view of a distal end portion of the ultrasonic endoscope shown in FIG. 1.

FIG. 3 is an explanatory diagram showing an optical axis of an imaging unit and an observation viewing range.

FIG. 4 is a side view of a distal end member.

FIGS. 5A to 5C are explanatory diagrams showing preferred configurations of the distal end member.

FIG. 6 is an enlarged view of a balloon groove.

FIG. 7 is a diagram showing a positional relationship between the imaging unit and an elevator unit in a case of being viewed from a Z(+) direction side.

FIG. 8 is a diagram showing a positional relationship between the imaging unit and a second pipe line member.

### DESCRIPTION OF THE PREFERRED EMBODIMENTS

Hereinafter, an embodiment of the present invention will be described with reference to the accompanying drawings.

Overall Configuration of Ultrasonic Endoscope

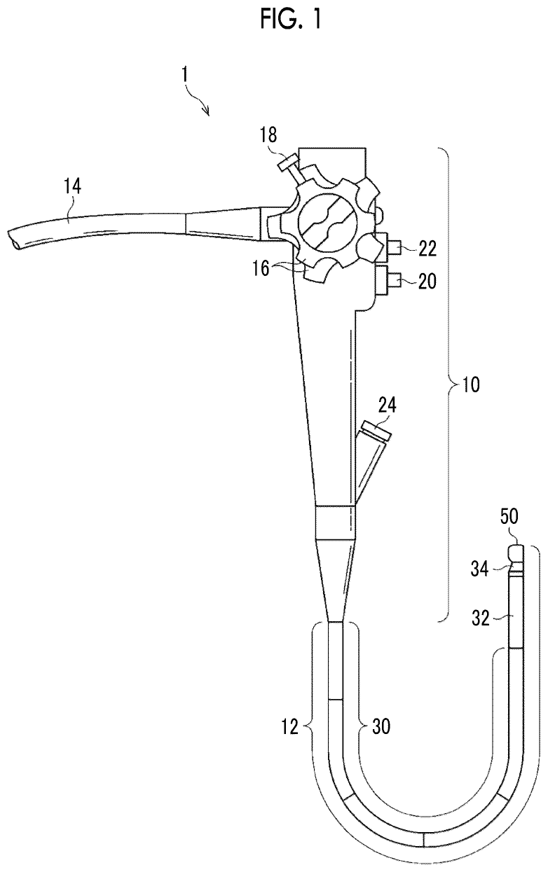

FIG. 1 is an overall configuration diagram of an ultrasonic endoscope (hereinafter, abbreviated as an “endoscope”) 1 according to the embodiment of the present invention.

As shown in FIG. 1, the endoscope 1 according to the present embodiment comprises an operating part 10 that is gripped by an operator to perform various operations, an insertion part 12 that is inserted into a body cavity of a patient, and a universal cord 14. The endoscope 1 is connected to a system constituent device including a processor device and a light source device (not shown) through the universal cord 14.

The operating part 10 is provided with various operation members operated by the operator and, for example, is provided with a pair of angle knobs 16, an elevating operation lever 18, an air and water supply button 20, a suction button 22, and the like.

In addition, a treatment tool inlet port 24 is provided on a distal end side of the operating part 10. The treatment tool introduced from the treatment tool inlet port 24 is inserted into a treatment tool insertion channel inserted into the insertion part 12.

The insertion part 12 extends from a distal end of the operating part 10 along a longitudinal axis direction, and the entire insertion part 12 is formed to have a small diameter and a long shape. The insertion part 12 includes a soft portion 30, a bendable portion 32, and a distal end portion 34 in order from a base end side to a distal end side. It should be noted that the insertion part 12 is an example of an insertion part according to the embodiment of the present invention, and the distal end portion 34 is an example of a distal end portion according to the embodiment of the present invention.

The soft portion 30 occupies most of the insertion part 12 from the base end side and has enough flexibility to be bent in any direction. In a case in which the insertion part 12 is inserted into the body cavity, the soft portion 30 is bent along an insertion route in the body cavity.

The bendable portion 32 is bent in an up-down direction and a left-right direction by a rotational operation of the pair of angle knobs 16 of the operating part 10. The distal end portion 34 can be directed in a desired direction by performing the bending operation of the bendable portion 32.

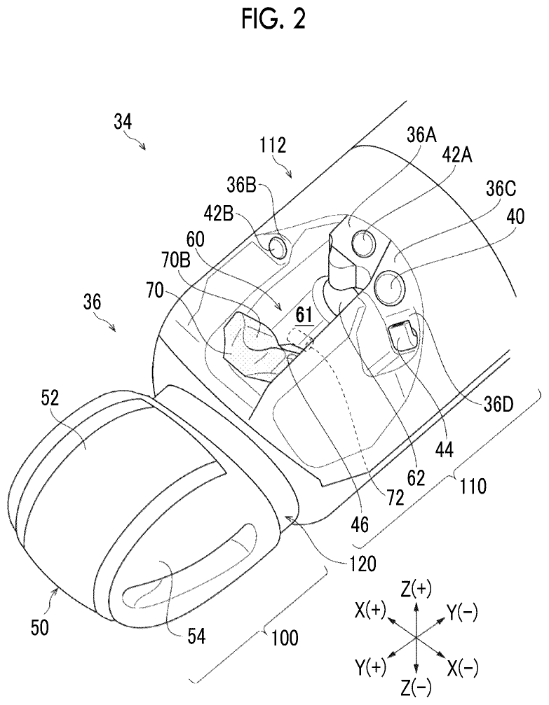

The distal end portion 34 comprises a distal end member 36 (see FIG. 2) described later. The distal end member 36 is disposed on the distal end side of the insertion part 12. The distal end member 36 includes an ultrasonic observation portion 100 on the distal end side thereof and an endoscope observation portion 110 on the base end side of the ultrasonic observation portion 100.

The universal cord 14 shown in FIG. 1 encompasses built-in components such as an ultrasonic cable, an electric cable, a light guide, and a fluid tube. A connector is provided at an end portion of the universal cord 14 (not shown), and by connecting the connector to the system constituent device, a control signal, power, illumination light, liquid, gas, and the like necessary for the operation of the endoscope 1 are supplied from the system constituent device to the endoscope 1. It should be noted that, in addition to the built-in components, a built-in component such as a treatment tool insertion channel, a bending operation wire, and an elevator operation wire is inserted into the insertion part 12.

Data of an ultrasonic image and data of an endoscopic image acquired by the endoscope 1 (ultrasonic observation portion 100 and endoscope observation portion 110) are each transmitted from the endoscope 1 to the system constituent device. Each data transmitted to the system constituent device is processed by the system constituent device and then displayed on a monitor (not shown) as the endoscopic image and the ultrasonic image, respectively.

Next, a configuration of the distal end portion 34 (distal end member 36) will be described with reference to FIG. 2. FIG. 2 is a perspective view showing an appearance of the distal end member 36, and shows a state in which an elevator 70 to be described later is located at a falling position.

Hereinafter, in a case of describing the configuration of each portion of the distal end member 36, a three-dimensional orthogonal coordinate system of X, Y, and Z will be used for convenience of description. In the drawings, a Z direction indicates an up-down direction, a Z(+) direction side indicates an up direction, and a Z(−) direction side indicates a down direction. In addition, in the drawings, an X direction indicates a direction perpendicular to the Z direction, an X(+) direction side indicates a left direction, and an X(−) direction side indicates a right direction. In addition, in the drawings, a Y direction indicates a direction perpendicular to both the Z direction and the X direction, a Y(+) direction side indicates a distal end side direction, and a Y(−) direction side indicates a base end side direction. It should be noted that each of the above-described directions indicates a direction in a case in which the distal end member 36 is viewed from the distal end side and an ultrasonic wave transmission and reception surface 52 of an ultrasonic transducer 50 described later is directed upward.

The Y direction corresponds to a longitudinal axis direction of the insertion part according to the embodiment of the present invention. In addition, the Z direction corresponds to a first direction according to the embodiment of the present invention, the Z(+) direction side corresponds to one side in the first direction according to the embodiment of the present invention, and the Z(−) direction side corresponds to the other side in the first direction according to the embodiment of the present invention. The X direction orthogonal to each of the Y direction (longitudinal axis direction) and the Z direction (first direction) corresponds to a second direction according to the embodiment of the present invention.

As shown in FIG. 2, the distal end member 36 includes the ultrasonic observation portion 100 and the endoscope observation portion 110 on the Y(−) direction side (base end side) of the ultrasonic observation portion 100.

The ultrasonic observation portion 100 and the endoscope observation portion 110 are consecutively installed through a balloon groove 120 formed between the ultrasonic observation portion 100 and the endoscope observation portion 110. A balloon that covers the ultrasonic transducer 50 is attached to the balloon groove 120. The balloon groove 120 is formed over an entire circumferential direction around the Y direction (longitudinal axis direction). The balloon groove 120 is an example of a balloon groove according to the embodiment of the present invention.

The ultrasonic observation portion 100 includes the ultrasonic transducer 50, and the ultrasonic transducer 50 is held by a housing 54.

The ultrasonic transducer 50 is a convex type in which a plurality of ultrasonic oscillators are arranged in an arc shape along the Y direction (longitudinal axis direction). The ultrasonic transducer 50 is configured such that an upper side surface (surface on the Z(+) direction side) thereof is formed as an ultrasonic wave transmission and reception surface 52, and emits an ultrasonic wave from the ultrasonic wave transmission and reception surface 52 toward the Z(+) direction side (one side in the first direction). Specifically, in a case of being viewed in the X direction, the ultrasonic waves emitted from the ultrasonic wave transmission and reception surface 52 (plurality of ultrasonic oscillators) are fan-shaped scanned (convex-scanned) obliquely rearward (Y(−) direction side and Z(+) direction side) to obliquely forward (Y(+) direction side and Z(+) direction side). The emission direction of the ultrasonic waves scanned in this way includes at least a component on the Z(+) direction side. It should be noted that it is sufficient that the emission direction of most of the ultrasonic waves to be scanned include the component on the Z(+) direction side, and the emission direction of some ultrasonic waves need not include the component on the Z(+) direction side (for example, in a case of the Y(+) direction side or in a case in which the direction includes a component on the Z(−) direction side). Data for generating the ultrasonic image is acquired by the ultrasonic transducer 50 configured in this manner. It should be noted that the ultrasonic transducer 50 is provided at the distal end portion 34 and is an example of an ultrasonic transducer according to the embodiment of the present invention.

The endoscope observation portion 110 includes a body member 112 formed in a substantially cylindrical shape. The body member 112 is made of an insulating material having insulating properties, for example, a resin material such as plastic materials such as a methacrylic resin, a polyphenyl sulfone resin, a polyether imide resin, a polyether ether ketone resin, and a polycarbonate.

The body member 112 includes an observation window 40 for observing an inside of a subject, illumination windows 42A and 42B for illuminating the inside of the subject, and an air and water supply nozzle 44 for cleaning the observation window 40 and the like. The observation window 40 is disposed on the Y(−) direction side (base end side) with respect to the elevator 70.

The body member 112 includes an elevator housing portion 60 that houses the elevator 70 therein. The elevator housing portion 60 includes an opening 46 having a rectangular shape in plan view in a case of being viewed from the Z(+) direction side. The opening 46 is formed to be open toward the Z(+) direction side in the body member 112. An opening direction of the opening 46 in the present embodiment is a direction (that is, a direction perpendicular to an X-Y plane) including only a component on the Z(+) direction side in a case of being viewed in the X direction. It should be noted that the opening direction of the opening 46 need only be a direction including at least the component on the Z(+) direction side, and may be, for example, a direction (that is, a direction including both components on the Z(+) direction side and the Y(+) direction side), which is obliquely forward in a case of being viewed in the X direction.

A treatment tool such as a biopsy needle is led out of the distal end member 36 from the opening 46. This treatment tool is led out toward an ultrasonic wave scanning range of the ultrasonic transducer 50. In addition, the elevator housing portion 60 is provided on the Z(−) direction side (lower side) of the opening 46.

A treatment tool outlet port 62 that communicates with an elevator housing space 61 of the elevator housing portion 60 is provided on the Y(−) direction side (base end side) of the elevator housing portion 60. The treatment tool outlet port 62 communicates with the treatment tool inlet port 24 of the operating part 10 via the treatment tool insertion channel inserted and disposed into the insertion part 12 (see FIG. 1). As a result, the treatment tool introduced from the treatment tool inlet port 24 is guided to the elevator housing space 61 from the treatment tool outlet port 62 via the treatment tool insertion channel.

The elevator 70 is housed in the elevator housing space 61 of the elevator housing portion 60. The elevator 70 is disposed on the Y(−) direction side with respect to the ultrasonic transducer 50. The elevator 70 is provided in a rotationally movable manner about a rotational movement shaft 72 disposed along the X direction, and is rotationally moved between an elevating position and a falling position. The elevating position refers to a position of the elevator 70 (that is, a position at which the elevator 70 is in a state of being elevated toward the Z(+) direction side) in a case in which the distal end side (opposite side to the rotational movement shaft 72) of the elevator 70 is moved to an end position on the Y(−) direction side in a rotationally movable range of the elevator 70. The falling position refers to a position of the elevator 70 (that is, a position at which the elevator 70 is in a state of being fallen toward the Y(+) direction side) in a case in which the distal end side of the elevator 70 is moved to an end position on the Y(+) direction side in the rotationally movable range of the elevator 70.

The elevator 70 is made of a metal material such as stainless steel, and includes a treatment tool guide surface 70B on the upper surface side. The treatment tool guided to the elevator housing portion 60 is guided along the treatment tool guide surface 70B, and is led out of the opening 46 of the elevator housing portion 60.

In a case in which the elevating operation lever 18 (see FIG. 1) is operated, the elevator 70 configured as described above operates (elevating and falling operation) between the elevating position and the falling position about the rotational movement shaft 72. For example, a leading-out direction (leading-out angle) of the treatment tool guided by the treatment tool guide surface 70B of the elevator 70 and led out from the opening 46 can be changed by operating the elevator 70 by the operation of the elevating operation lever 18 and adjusting an elevating angle of the elevator 70 from the falling position. It should be noted that the body member 112 is provided with an elevator unit which will be described later. The elevator unit is a unit in which a plurality of components (elevator 70, case member 402, and the like) constituting the elevator housing portion 60 are integrated.

As shown in FIG. 2, the illumination windows 42A and 42B are disposed respectively on illumination window disposition surfaces 36A and 36B provided on the body member 112. Inside the illumination windows 42A and 42B, light emission portions constituting an illumination unit are housed. These light emission portions are connected to a light source device via a light guide. The illumination light transmitted from the light source device through the light guide is emitted from the light emission portions and to irradiate a treatment target part through the illumination windows 42A and 42B.

The observation window 40 is disposed on an observation window disposition surface 36C provided in the body member 112. An imaging system unit including an imaging optical system, a solid-state imaging element, a circuit board, and a signal cable, which constitute an imaging unit (camera unit), is housed in the observation window 40. The observation window 40 and the imaging system unit constitute the imaging unit. Accordingly, light (reflected light) from the treatment target part (subject) irradiated by the illumination windows 42A and 42B is taken in from the observation window 40, and the light is imaged as an observation image on the solid-state imaging element through the imaging optical system. Data for generating the endoscopic image is acquired by the imaging unit.

The air and water supply nozzle 44 is disposed on a nozzle disposition surface 36D provided on the body member 112. In a case in which the air and water supply button 20 (see FIG. 1) is operated, a cleaning liquid such as water or air (fluid) is jetted from the air and water supply nozzle 44 toward the observation window 40 or the like, to clean the observation window 40 or the like.

### Description of Ultrasonic Endoscope of Present Disclosure

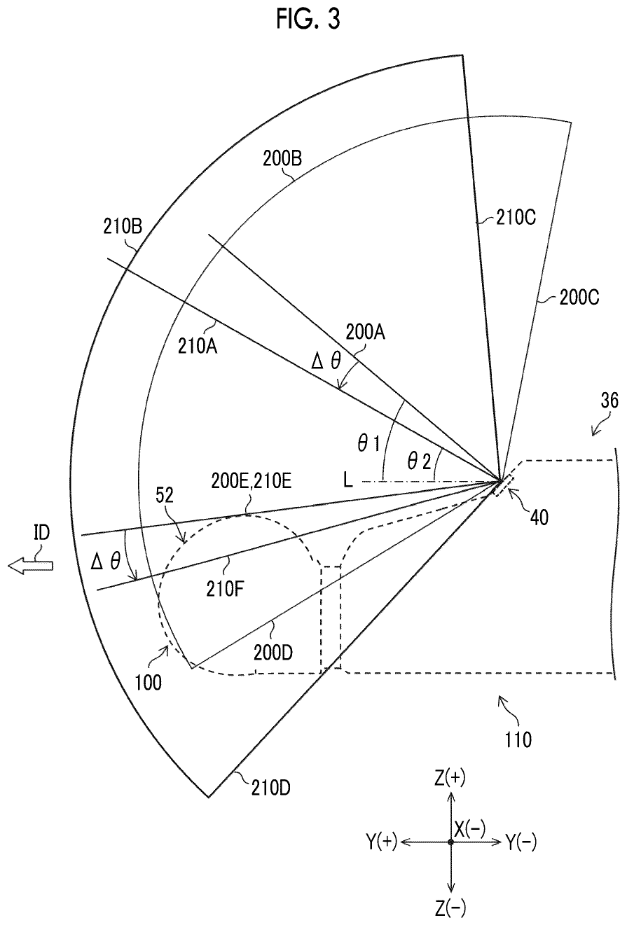

Next, a principle applied to the ultrasonic endoscope according to the present disclosure will be described with reference to FIG. 3. FIG. 3 is a diagram in which the distal end member 36 of the endoscope 1 is modeled, which is viewed from the X(−) direction side, and is a diagram showing a comparison between a case (comparative example) in which the imaging unit includes an optical axis 200A and a case (example) in which the imaging unit includes an optical axis 210A having a different inclined angle from the optical axis 200A. As shown in FIG. 3, the distal end member 36 comprises the ultrasonic observation portion 100 and the endoscope observation portion 110.

Here, the optical axis (200A or 210A) of the imaging unit corresponds to the optical axis of the observation window 40, and is a straight line that passes through the center of the observation window 40 and that extends in the normal direction of the surface of the observation window 40. In addition, hereinafter, in a case of being viewed from the X(−) direction side, an angle (inclined angle) formed between the optical axis (200A or 210A) of the imaging unit and a reference axis L parallel to the Y direction is referred to as an “angle of view”, and a direction indicated by the optical axis is referred to as a “viewing direction”. In addition, an observation viewing range (200B or 210B) in the imaging unit (observation window 40) is a range having a certain width centered on each optical axis (200A or 210A), and the observation viewing ranges of both are ranges having the same width. For example, the observation viewing range 200B corresponding to the optical axis 200A of the imaging unit is a fan-shaped range surrounded by a first end 200C on one side and a second end 200D on the other side with respect to the optical axis 200A.

For example, in a case in which the imaging unit having the optical axis 200A as shown in FIG. 3 is applied, the following points are described as characteristics or problems of the ultrasonic endoscope 1.

First, the ultrasonic wave transmission and reception surface 52 of the ultrasonic observation portion 100 is formed as a curved surface that is convex toward the Z(+) direction side (upper side) in a case of being viewed from the X(−) direction side. Therefore, as shown in FIG. 3, in the imaging unit having the optical axis 200A, the ultrasonic observation portion 100 is reflected in the observation viewing range 200B. The range in which the ultrasonic observation portion 100 is reflected is a range surrounded by the second end 200D and a tangent 200E. Here, the tangent 200E is a straight line that passes through the center of the observation window 40 and that is in contact with the ultrasonic wave transmission and reception surface 52.

Second, the viewing direction (direction indicated by the optical axis 200A) of the imaging unit and an insertion direction ID (Y direction) of the endoscope 1 do not match each other. That is, the optical axis 200A of the imaging unit is directed in a direction inclined by an angle of view θ1 with respect to the insertion direction ID. Therefore, in a case in which the operator inserts the endoscope 1 while viewing the endoscopic image, the ultrasonic observation portion 100 is easily pressed against the subject.

Therefore, in the endoscope 1, it is required to bring the viewing direction of the imaging unit closer to the direct vision, that is, to reduce the angle of view which is the inclined angle of the optical axis 200A. Then, in order to bring the viewing direction of the imaging unit closer to the direct vision, it is considered to change the imaging unit (observation window 40) to the imaging unit having the optical axis 210A having the angle of view (inclined angle with respect to the reference axis L described above) smaller than that of the optical axis 200A as shown in FIG. 3. That is, an angle of view θ2 of the optical axis 210A after the change is smaller than the angle of view θ1 of the optical axis 200A before the change by a difference Δθ, and the observation viewing range 210B in a case in which the optical axis 210A after the change is provided and the observation viewing range 200B in a case in which the optical axis 200A before the change is provided are different from each other only in the inclination around the X direction and have the same range. It should be noted that the observation viewing range 210B is a range surrounded by the first end 210C on one side and the second end 210D on the other side with respect to the optical axis 210A. Accordingly, the imaging unit (observation window 40) having the optical axis 210A can be brought closer to the direct vision.

On the other hand, in a case in which the viewing direction (optical axis direction) in the imaging unit is changed as described above, there is a concern that the endoscopic image in a desired range (range including the treatment target part) that is originally required cannot be obtained because the range (range surrounded by the tangent 210E and the second end 210D) in which the ultrasonic observation portion 100 is reflected in the observation viewing range 210B becomes relatively larger as the viewing direction is changed.

In consideration of the above-described concern, the present inventors have conducted studies to make a range in which the ultrasonic observation portion 100 is reflected in the observation viewing range 210B after the change of the viewing direction in the imaging unit equal to a range of the ultrasonic observation portion 100 reflected in the observation viewing range 200B before the change of the viewing direction, thereby completing the present invention.

The angle of view θ2 of the optical axis 210A after the change in the viewing direction is inclined by the difference Δθ from the optical axis 200A before the change in the viewing direction because the viewing direction of the imaging unit is brought closer to the direct vision. Therefore, in the present embodiment, the ultrasonic observation portion 100 is disposed at a position shifted to the Z(−) direction side by an amount corresponding to the difference Δθ. In this case, an angle formed between a tangent 210F (straight line that passes through the center of the observation window 40 and that is in contact with the ultrasonic wave transmission and reception surface 52) with the ultrasonic observation portion 100 after the ultrasonic observation portion 100 is shifted to the Z(−) direction side and the tangent 210E before the ultrasonic observation portion 100 is shifted to the Z(−) direction side is the difference Δθ. As a result, the ultrasonic observation portion 100 that is reflected in the observation viewing range 210B after the change of the viewing direction is equivalent to the range in which the ultrasonic observation portion 100 is reflected in the observation viewing range 200B before the change of the viewing direction. That is, a reflected glare range of the ultrasonic observation portion 100 can be made constant.

In addition, as a method of shifting the ultrasonic observation portion 100 to the Z(−) direction side, for example, it is also considered to enlarge both the ultrasonic observation portion 100 and the endoscope observation portion 110 as a whole to the Z(−) direction side, and lower the position of the ultrasonic observation portion 100 on the Z(−) direction side to the Z(−) direction side. However, in a case in which such a configuration is adopted, the endoscope observation portion 110 is also increased in diameter by an amount of magnification to the Z(−) direction side, and thus the diameter of the entire distal end portion including the distal end member 36 is increased. On the other hand, in the present embodiment, since only the ultrasonic observation portion 100 is disposed to be shifted to the Z(−) direction side, it is possible to avoid the increase in diameter of the distal end portion.

### Embodiment

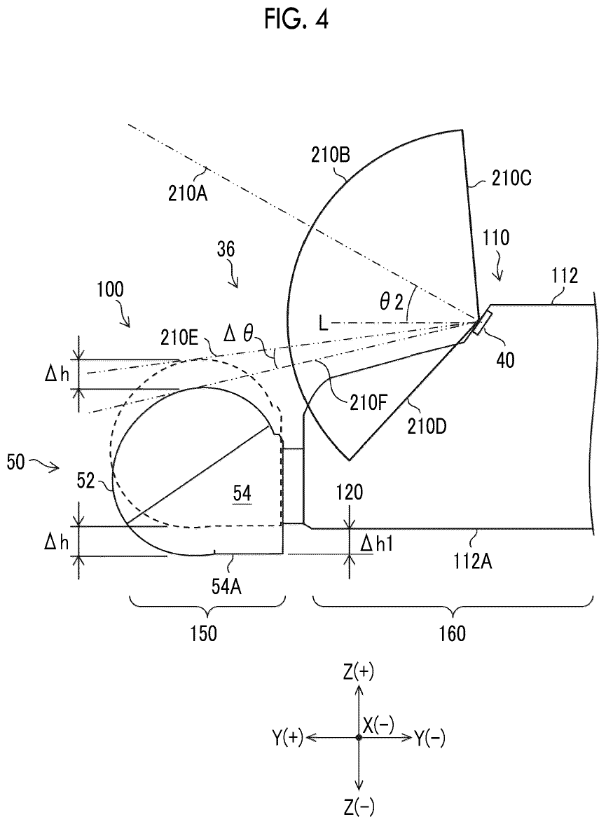

Hereinafter, the distal end member 36 of the endoscope 1 according to the present embodiment will be described in detail. FIG. 4 is a side view in a case in which the distal end member 36 is viewed from the X(−) direction side. As shown in FIG. 4, the distal end member 36 includes a first region 150 including the ultrasonic observation portion 100 and a second region 160 including the endoscope observation portion 110. The second region 160 is disposed on the Y(−) direction side (base end side) with respect to the first region 150. The first region 150 and the second region 160 are examples of a first region and a second region according to the embodiment of the present invention, respectively.

The ultrasonic observation portion 100 includes the housing 54, and the housing 54 includes a bottom surface portion 54A on the Z(−) direction side. The endoscope observation portion 110 includes the body member 112, and the body member 112 includes a bottom surface portion 112A on the Z(−) direction side. The bottom surface portion 54A is an example of a first bottom surface portion of the first region according to the embodiment of the present invention, and the bottom surface portion 112A is an example of a second bottom surface portion of the second region according to the embodiment of the present invention.

The optical axis 210A of the imaging unit is inclined in the direct vision direction (Y(+) direction side) by the difference 40 with respect to the angle of view θ1 (inclined angle with respect to the reference axis L of the optical axis 200A) of the imaging unit in the above-described comparative example, and the angle of view θ2 is (θ1−Δθ). In response to the difference Δθ, only the ultrasonic observation portion 100 of the ultrasonic observation portion 100 and the endoscope observation portion 110 is disposed to be shifted by a predetermined distance Δh to the Z(−) direction side such that the tangent to the ultrasonic wave transmission and reception surface 52 is in a state indicated by a reference numeral 210E to a reference numeral 210F. It should be noted that the angle formed between the tangent 210F and the tangent 210E is the difference Δθ. In this way, since the ultrasonic observation portion 100 is disposed to be shifted only to the Z(−) direction side, the bottom surface portion 54A in the first region 150 is disposed to be shifted to the Z(−) direction side by Δh1 with respect to the bottom surface portion 112A in the second region 160.

According to the present embodiment, since the bottom surface portion 112A in the second region 160 is located on the Z(+) direction side by Δh1 with respect to the bottom surface portion 54A in the first region 150 by adopting the above-described disposition configuration, it is possible to avoid the increase in diameter of the second region 160 including the endoscope observation portion 110. As a result, it is possible to keep the range of the ultrasonic observation portion 100 reflected in the observation viewing range constant without increasing the diameter of the distal end portion 34.

In addition, according to the present embodiment, the viewing direction in the imaging unit can be brought closer to the direct vision. The angle of view θ2 of the optical axis 210A of the imaging unit is, for example, less than 40 degrees. The angle of view θ2 is preferably 35 degrees or less, and more preferably 25 degrees or less. On the other hand, in consideration of the distance at which the ultrasonic observation portion 100 can be disposed to be shifted to the Z(−) direction side, the angle of view θ2 is preferably 15 degrees or more and more preferably 20 degrees or more.

### Preferred Embodiments

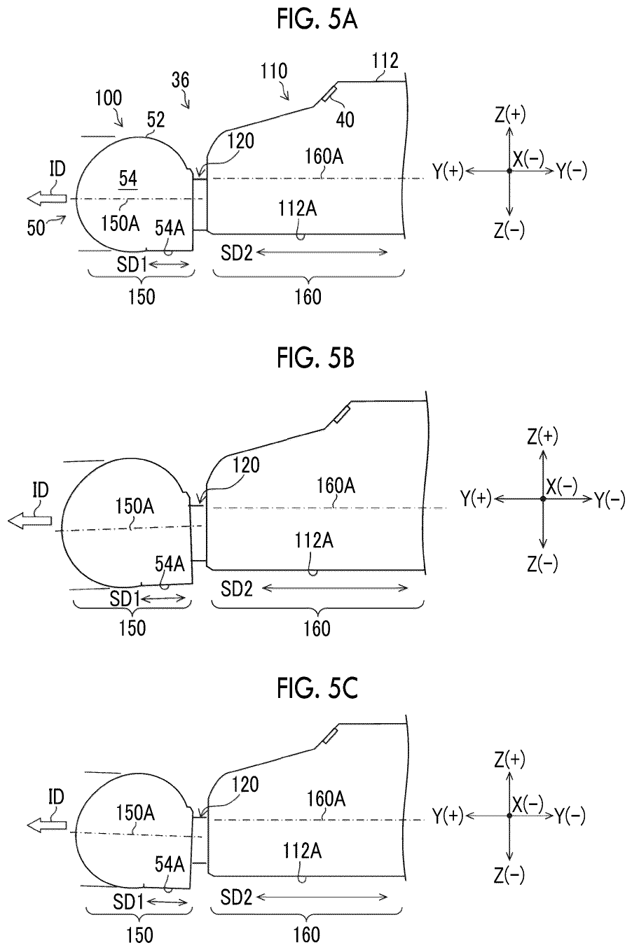

Next, some preferred embodiments of the distal end member 36 of the present example will be described with reference to FIGS. 5A to 8.

### Disposition Relationship Between First Region 150 and Second Region 160

FIGS. 5A to 5C are explanatory diagrams of the distal end member 36 in a case of being viewed from the X(−) direction side. As shown in FIGS. 5A to 5C, the first region 150 including the ultrasonic observation portion 100 and the second region 160 including the endoscope observation portion 110 are disposed with the balloon groove 120 interposed therebetween. In addition, as described above, the bottom surface portion 54A of the first region 150 is located on the Z(−) direction side with respect to the bottom surface portion 112A of the second region 160. A preferable disposition relationship between the first region 150 and the second region 160 will be described based on respective central axes 150A and 160A.

Here, the central axis 150A is a straight line that passes through the center between the bottom surface portion 54A and a highest point (height direction) of the ultrasonic wave transmission and reception surface 52, and that is parallel to a plane direction SD1 of the bottom surface portion 54A. In addition, the central axis 160A is a straight line that passes through the center of the bottom surface portion 112A and a highest point (height direction) of the body member 112, and that is parallel to a plane direction SD2 of the bottom surface portion 112A. The central axis 150A and the central axis 160A are examples of a first central axis of the first region and a second central axis of the second region according to the embodiment of the present invention, respectively.

In the distal end portion 34, it is preferable that the angle formed between the central axis 150A of the first region 150 and the central axis 160A of the second region 160 is 1 degree or less. By setting the angle to 1 degree or less, in a case in which the distal end portion 34 is inserted into the body cavity, the first region 150 including the ultrasonic observation portion 100 can be prevented from being pressed against the body cavity wall.

FIGS. 5A, 5B, and 5C show examples of the angle formed between the central axis 150A and the central axis 160A, and the respective angles are different from each other. In FIGS. 5A to 5C, the angle is defined as an angle of the central axis 150A with respect to the central axis 160A, the central axis 160A being parallel to the Y direction.

In FIG. 5A, the central axis 150A and the central axis 160A are each parallel to the Y direction. In this configuration, the angle formed between the central axis 150A and the central axis 160A is 0 degrees.

In FIG. 5B, the central axis 150A is inclined with respect to the Y direction, and the Y(+) direction side is lower than the Y(−) direction side in the Z direction. In this configuration, the angle of the central axis 150A relative to the central axis 160A is 1 degree.

In FIG. 5C, the central axis 150A is inclined with respect to the Y direction, and the Y(+) direction side is higher than the Y(−) direction side in the Z direction. In this configuration, the angle of the central axis 150A relative to the central axis 160A is 1 degree.

In all of FIGS. 5A, 5B, and 5C, the angle between the central axis 150A and the central axis 160A is 1 degree or less.

Then, a preferable relationship between the insertion direction ID and the plane direction SD1 of the bottom surface portion 54A will be described. The insertion direction ID is a direction in which the distal end portion 34 is inserted into the body cavity. The plane direction SD1 of the bottom surface portion 54A is a direction extending along the Y direction as indicated by an arrow. It is preferable that the angle formed between the insertion direction ID and the plane direction SD1 of the bottom surface portion 54A is 1 degree or less. By setting the angle to 1 degree or less, in a case in which the distal end portion 34 is inserted into the body cavity, it is possible to avoid the ultrasonic wave transmission and reception surface 52 or the bottom surface portion 54A from being pressed against the body cavity wall.

It should be noted that, in FIGS. 5A, 5B, and 5C, the angle between the insertion direction ID and the plane direction SD1 of the bottom surface portion 54A is 1 degree or less. FIGS. 5A to 5C show a case in which the central axis 150A and the plane direction SD1 of the bottom surface portion 54A are parallel to each other, but the central axis 150A and the plane direction SD1 of the bottom surface portion 54A need not be parallel to each other.

### Balloon Groove 120

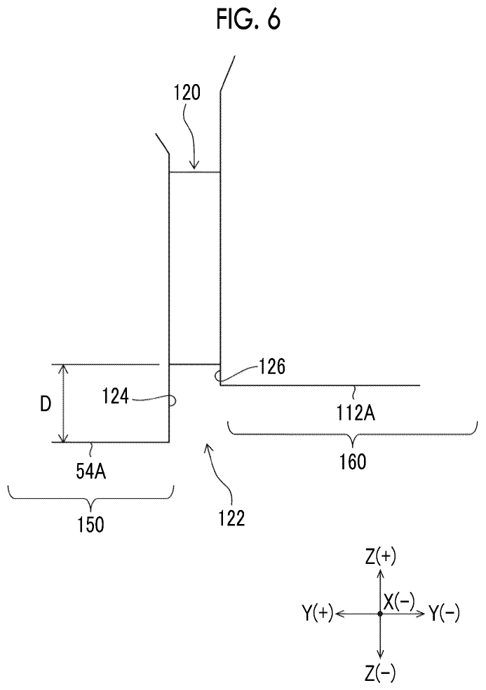

FIG. 6 is an enlarged view of the balloon groove 120 shown in FIG. 4 in a case of being viewed from the X(−) direction side. As shown in FIG. 6, a groove region 122 on the Z(−) direction side of the balloon groove 120 is formed of a pair of groove side surface portions 124 and 126 facing each other the Y direction. Then, among the pair of groove side surface portions 124 and 126, the groove side surface portion 124 is provided on the Y(−) direction side (base end side) of the first region 150, the groove side surface portion 126 is provided on the Y(+) direction side (distal end side) of the second region 160, and the groove side surface portion 124 is formed to extend to the Z(−) direction side with respect to the groove side surface portion 126. It should be noted that the groove side surface portion 124 and the groove side surface portion 126 are examples of a first groove side surface portion and a second groove side surface portion according to the embodiment of the present invention, respectively.

In the attachment of the balloon to the distal end portion 34, in the convex type endoscope 1, since the ultrasonic wave transmission and reception surface 52 is formed of a surface curved in the Z(+) direction side (upper side), a depth of the upper side of the balloon groove 120 does not affect the attachment of the balloon. On the other hand, since the bottom surface portion 54A on the Z(−) direction side (lower side) of the housing 54 is formed of a substantially flat surface, a depth of the balloon groove 120 on the lower side is shallow, and it is difficult to attach the balloon.

However, in the present example, by disposing the bottom surface portion 54A on the Z(−) direction side with respect to the bottom surface portion 112A, the groove side surface portion 124 can be extended on the Z(−) direction side with respect to the groove side surface portion 126. As a result, a depth D of the balloon groove 120 on the Z(−) direction side can be increased, and the attachability of the balloon is improved.

### Disposition relationship between Imaging Unit 300 and Elevator Unit 400

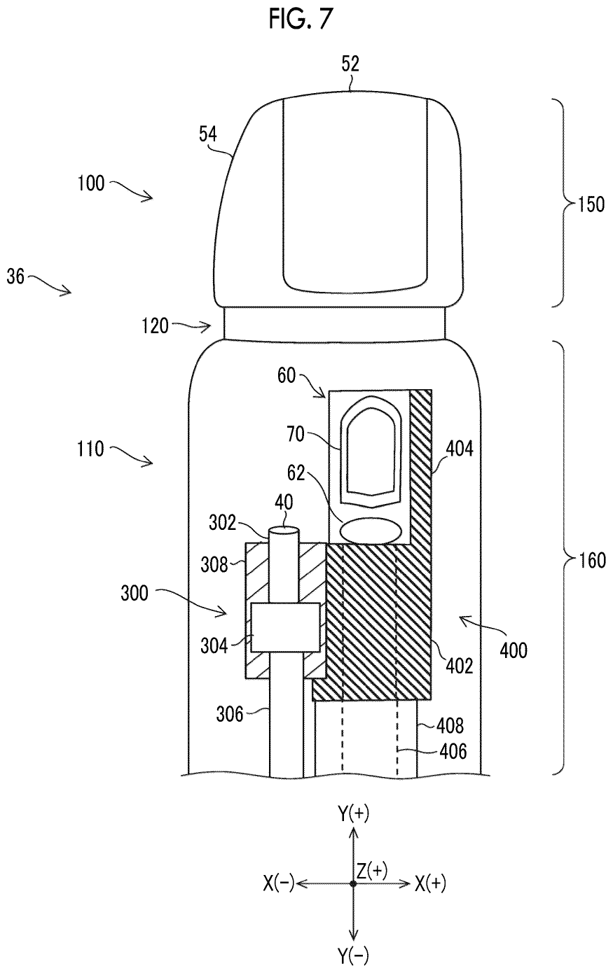

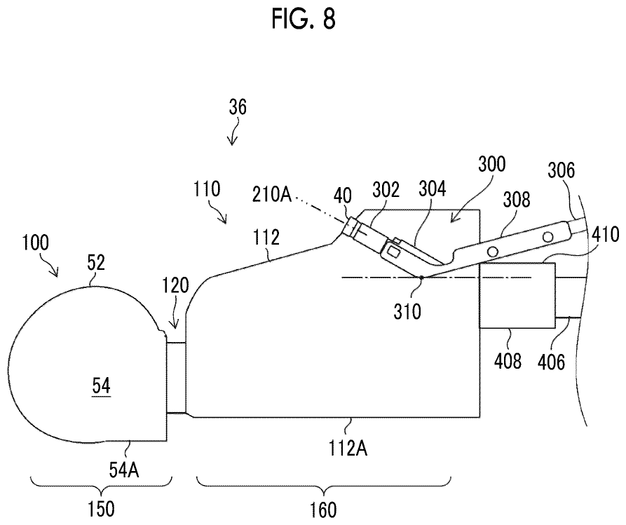

FIG. 7 is a diagram showing a positional relationship between the imaging unit 300 and the elevator unit 400 in a case of being viewed from the Z(+) direction side. FIG. 8 is a diagram showing a positional relationship between the imaging unit 300 and a second pipe line member 408.

As shown in FIG. 7, the imaging unit 300 and the elevator unit 400 are disposed in the second region 160.

First, the imaging unit 300 will be described. The imaging unit 300 includes a lens barrel 302 including the observation window 40, a large diameter portion 304, and a signal cable 306 in this order from the Y(+) direction side to the Y(−) direction side. The lens barrel 302 is a tubular member having a narrower width than the large diameter portion 304, which will be described later, in the X direction, and includes an imaging lens for imaging an observation target therein. The configuration of the imaging lens is not particularly limited. The large diameter portion 304 is a portion having a larger width than the lens barrel 302 in the X direction. The large diameter portion 304 includes a prism, a solid-state imaging element, and a circuit board therein. The signal cable 306 is electrically connected to the solid-state imaging element via the circuit board. The signal cable 306 is formed by, for example, bundling a large number of signal wires, and has flexibility.

The second region 160 includes a holder 308 that houses the imaging unit 300. The holder 308 houses the lens barrel 302, the large diameter portion 304, and the signal cable 306, and protects the lens barrel 302, the large diameter portion 304, and the signal cable 306. The holder 308 may house a part of the lens barrel 302, the large diameter portion 304, and the signal cable 306.

The holder 308 is formed by bending one elongated metal plate. The holder 308 has, for example, a U-shape in a cross-sectional view in the XZ plane, and includes a bottom portion that extends in the Y direction and wall portions that extend to the Z(+) direction side from both edges of the bottom portion. The holder 308 has a V-shape that extends to the Z(+) direction side along the lens barrel 302 and the signal cable 306 in a case of being viewed from the X(−) direction side. The bent portion 310 of the holder 308 having a V-shape is located on the Z(−) direction side in the holder 308. The holder 308 is an example of an imaging unit housing portion according to the embodiment of the present invention. The bent portion 310 is an example of a position of an outermost end portion side on the other side in the first direction according to the embodiment of the present invention.

Then, the elevator unit 400 will be described. The elevator unit 400 is located on the X(+) direction side with respect to the imaging unit 300 in a case of being viewed from the Z(+) direction side. The elevator unit 400 includes the elevator 70 and the case member 402. The case member 402 is made of, for example, a metal material having corrosion resistance. The elevator 70 is provided on the case member 402 in a rotationally movable manner. The case member 402 includes a side wall 404, and a lever (not shown) to which an operation wire is connected is housed in the side wall 404. The lever and the elevator 70 are connected to each other via the rotational movement shaft 72. The lever rotationally moves the elevator 70 about the rotational movement shaft 72 in response to the operation of the elevating operation lever 18.

The elevator housing space 61 (see FIG. 2) of the elevator housing portion 60 is defined by the case member 402. The case member 402 is provided with the treatment tool outlet port 62 that communicates with the elevator housing space 61. The case member 402 is provided with a first pipe line member 406 and the second pipe line member 408 that constitute the treatment tool insertion channel on the Y(−) direction side (base end side). The end portion of the second pipe line member 408 on the Y(+) direction side (distal end side) is externally fitted to the end portion of the first pipe line member 406 on the Y(−) direction side (base end side), so that the first pipe line member 406 and the second pipe line member 408 are connected to each other. As a result, the second pipe line member 408 communicates with the treatment tool outlet port 62 via the first pipe line member 406. It should be noted that an upper surface 410 of the second pipe line member 408 on the Z(+) direction side is a position on the outermost end portion side on one side in the first direction. The elevator unit 400, the case member 402, and the second pipe line member 408 are examples of an elevator unit, a case member, and a pipe member according to the embodiment of the present invention, respectively. The elevator housing space 61 is an example of a space portion according to the embodiment of the present invention.

As shown in FIG. 7, at least a part of a region of the holder 308 is disposed at a position overlapping with the elevator unit 400 in a case of being viewed from the Z(+) direction side. In addition, the holder 308 is located on the Z(+) direction side with respect to the elevator unit 400. Accordingly, the width of the endoscope observation portion 110 in the X direction in the second region 160 can be narrowed, and the distal end member 36 can be reduced in diameter.

As shown in FIG. 8, the position of the bent portion 310 of the holder 308 is located on the Z(−) direction side with respect to the upper surface of the second pipe line member 408 in a case of being viewed from the X(−) direction side. By bringing the observation window 40 closer to the Z(−) direction side with the bent portion 310 as a fulcrum, the viewing direction (optical axis 210A direction) in the imaging unit 300 can be brought closer to the direct vision direction. Further, since the base end side of the imaging unit 300 faces the Z(+) direction side, interference with the second pipe line member 408 can be avoided, the width of the endoscope observation portion 110 in the Z direction can be narrowed, and the distal end member 36 can be reduced in diameter.

Although the ultrasonic endoscope according to the present embodiment has been described above, the present invention may be improved or modified in some ways without departing from the gist of the present invention.

### EXPLANATION OF REFERENCES

1: ultrasonic endoscope
10: operating part
12: insertion part
14: universal cord
16: angle knob
18: elevating operation lever
20: air and water supply button
22: suction button
24: treatment tool inlet port
30: soft portion
32: bendable portion
34: distal end portion
36: distal end member
36A: illumination window disposition surface
36B: illumination window disposition surface
36C: observation window disposition surface
36D: nozzle disposition surface
42A: illumination window
42B: illumination window
44: air and water supply nozzle
46: opening
50: ultrasonic transducer
52: ultrasonic wave transmission and reception surface
54: housing
54A: bottom surface portion
60: elevator housing portion
62: treatment tool outlet port
70: elevator
70B: treatment tool guide surface
72: rotational movement shaft
100: ultrasonic observation portion
110: endoscope observation portion
112: body member
112A: bottom surface portion
120: balloon groove
122: groove region
124: groove side surface portion
126: groove side surface portion
150: first region
150A: central axis
160: second region
160A: central axis
200A: optical axis
200B: observation viewing range
200C: first end
200D: second end
200E: tangent
210A: optical axis
210B: observation viewing range
210C: first end
210D: second end
210E: tangent
210F: tangent
300: imaging unit
302: lens barrel
304: large diameter portion
306: signal cable
308: holder
310: bent portion
400: elevator unit
402: case member
404: side wall
406: first pipe line member
408: second pipe line member
410: upper surface

What is claimed is:

1. An ultrasonic endoscope comprising:
a distal end portion that is disposed on a distal end side of an insertion part extending along a longitudinal axis direction;
an ultrasonic transducer that is provided at the distal end portion and that emits an ultrasonic wave toward one side in a first direction orthogonal to the longitudinal axis direction; and
an imaging unit that is disposed on a base end side in the longitudinal axis direction with respect to the ultrasonic transducer and that images a subject,
wherein the distal end portion includes a first region in which the ultrasonic transducer is provided and a second region that is disposed on the base end side in the longitudinal axis direction with respect to the first region and in which the imaging unit is provided,
a first bottom surface portion of the first region and a second bottom surface portion of the second region are each disposed on the other side in the first direction, which is opposite to the one side, and
the first bottom surface portion is disposed on the other side in the first direction with respect to the second bottom surface portion.

2. The ultrasonic endoscope according to claim 1,
wherein, in a case of being viewed in a second direction orthogonal to each of the longitudinal axis direction and the first direction, an angle formed between a first central axis of the first region and a second central axis of the second region is 1 degree or less.

3. The ultrasonic endoscope according to claim 2,
wherein, in a case of being viewed in the second direction, an angle formed between a plane direction of the first bottom surface portion and the longitudinal axis direction is 1 degree or less.

4. The ultrasonic endoscope according to claim 1,
wherein the distal end portion includes a balloon groove that is provided between the first region and the second region and that is used for attaching a balloon that covers the ultrasonic transducer.

5. The ultrasonic endoscope according to claim 4,
wherein a groove region of the balloon groove on the other side in the first direction is formed of a pair of groove side surface portions facing each other in the longitudinal axis direction,
a first groove side surface portion that is one of the pair of groove side surface portions is provided on a base end side of the first region in the longitudinal axis direction, and a second groove side surface portion that is the other of the pair of groove side surface portions is provided on a distal end side of the second region in the longitudinal axis direction, and
the first groove side surface portion is formed to extend toward the other side in the first direction with respect to the second groove side surface portion.

6. The ultrasonic endoscope according to claim 1,
wherein the second region includes an elevator unit in which an elevator is provided in a rotationally movable manner.

7. The ultrasonic endoscope according to claim 6,
wherein the elevator unit includes the elevator and a case member that defines a space portion in which the elevator is housed.

8. The ultrasonic endoscope according to claim 7,
wherein the case member includes a treatment tool outlet port that is open to the space portion, and
the case member is provided with a pipe member that communicates with the treatment tool outlet port.

9. The ultrasonic endoscope according to claim 8, further comprising:
an imaging unit housing portion that houses the imaging unit,
wherein, in a case of being viewed in the first direction, at least a part of a region of the imaging unit housing portion is disposed at a position overlapping with the elevator unit.

10. The ultrasonic endoscope according to claim 9,
wherein, in a case of being viewed in a second direction orthogonal to each of the longitudinal axis direction and the first direction, at least a part of a region of the imaging unit housing portion is disposed at a position overlapping with the pipe member.

11. The ultrasonic endoscope according to claim 9,
wherein, in a case of being viewed in a second direction orthogonal to each of the longitudinal axis direction and the first direction, a position of an outermost end portion side of the imaging unit housing portion on the other side in the first direction is provided on the other side in the first direction with respect to a position of an outermost end portion side of the pipe member on the one side in the first direction.
